(19) United States
(12) Patent Application Publication
(10) Pub. No.: US 2003/0216800 A1
(43) Pub. Date: Nov. 20, 2003

(54) Implantable medical device conductor insulation and process for forming

(75) Inventors: Michael J. Ebert, Fridley, MN (US); John L. Sommer, Coon Rapids, MN (US); Jordon D. Honeck, Maple Grove, MN (US); Richard D. Ries, Stillwater, MN (US); Pedro A. Meregotte, Vadnais Heights, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/407,653

(22) Filed: Apr. 4, 2003

Related U.S. Application Data
(60) Provisional application No. 60/371,995, filed on Apr. 11, 2002.

(51) Int. Cl. A61N 1/05

(52) U.S. Cl. 607/122

(57) ABSTRACT

An implantable medical device that includes a lead body extending from a proximal end to a distal end, a plurality of conductors extending between the proximal end and the distal end of the lead body, and an insulative layer formed of a hydrolytically stable polyimide material surrounding the plurality of conductors. In one embodiment, the hydrolytically stable polyimide material is an SI polyimide material.

### RELATED APPLICATION

The present invention claims priority and other benefits from U.S. Provisional Patent Application Serial No. 60/371,995, filed Apr. 11, 2002, entitled “BIO-STABLE IMPLANTABLE MEDICAL DEVICE LEAD CONDUCTOR INSULATION AND PROCESS FOR FORMING”, incorporated herein by reference in its entirety.

### FIELD OF THE INVENTION

The present invention relates generally to implantable medical device leads for delivering therapy, in the form of electrical stimulation, and in particular, the present invention relates to conductor coil insulation in implantable medical device leads.

### BACKGROUND OF THE INVENTION

Implantable medical electrical leads are well known in the fields of cardiac stimulation and monitoring, including neurological pacing and cardiac pacing and cardioversion/defibrillation. In the field of cardiac stimulation and monitoring, endocardial leads are placed through a transvenous route to position one or more sensing and/or stimulation electrodes in a desired location within a heart chamber or interconnecting vasculature. During this type of procedure, a lead is passed through the subclavian, jugular, or cephalic vein, into the superior vena cava, and finally into a chamber of the heart or the associated vascular system. An active or passive fixation mechanism at the distal end of the endocardial lead may be deployed to maintain the distal end of the lead at a desired location.

Routing an endocardial lead along a desired path to a target implant site can be difficult and is dependent upon the physical characteristics of the lead. At the same time, as will be readily appreciated by those skilled in the art, it is highly desirable that the implantable medical lead insulation possess high dielelectric properties, and exhibit durable and bio-stable properties, flexibility, and reduced size.

In light of the foregoing, up to the present invention the need still existed in the prior art for a material which is suitable for use as an insulator for leads of implantable electrical devices, and which provides a biostable, durable, high dielectric insulator for electrical stimulating leads where minimum insulation coverage is required.

### BRIEF SUMMARY OF THE INVENTION

The present invention relates to an implantable medical device that includes a lead body extending from a proximal end to a distal end, a plurality of conductors extending between the proximal end and the distal end of the lead body, and an insulative layer formed of a hydrolytically stable polyimide material surrounding the plurality of conductors.

In another embodiment of the present invention, an implantable medical device includes a housing generating electrical signals for delivering cardiac therapy, a lead having a lead body extending from a proximal end to a distal end, the proximal end of the lead being insertable within a connector block of the housing and electrically coupling the housing and the lead, a plurality of conductors extending between the proximal end and the distal end of the lead body, and an insulative layer formed of a hydrolytically stable polyimide material surrounding the plurality of conductors.

In another embodiment of the present invention, an implantable medical device includes a lead body extending from a proximal end to a distal end, a plurality of conductors extending between the proximal end and the distal end of the lead body, and an insulative layer formed of a hydrolytically stable polyimide material surrounding the plurality of conductors, wherein the insulative layer is positioned about the plurality of conductors in multiple coats to form multiple layers and has a thickness of between approximately 0.0001 of an inch and approximately 0.0020 of an inch.

In another embodiment of the present invention, an implantable medical device includes a housing generating electrical signals for delivering cardiac therapy, a lead having a lead body extending from a proximal end to a distal end, the proximal end of the lead body being insertable within a connector block of the housing and electrically coupling the housing and the lead, a plurality of conductors extending between the proximal end and the distal end of the lead body, and an insulative layer formed of an SI polyimide material surrounding the plurality of conductors, wherein the insulative layer is positioned about the plurality of conductors in multiple coats to form multiple layers and has a thickness of between approximately 0.0001 inches and approximately 0.0050 inches.

In an embodiment of the present invention, the hydrolytically stable polyimide material is an SI polyimide material.

### BRIEF DESCRIPTION OF THE DRAWINGS

Other advantages and features of the present invention will be readily appreciated as the same becomes better understood by reference to the following detailed description when considered in connection with the accompanying drawings, in which like reference numerals designate like parts throughout the figures thereof and wherein:

FIG. 1 is a schematic diagram of an exemplary implantable medical device in accordance with the present invention;

FIG. 2 is a cross-sectional view of a lead of an implantable medical device according to the present invention, taken along cross-sectional lines II-II of FIG. 1;

FIG. 3 is a cross-sectional view of a lead of an implantable medical device according to the present invention, taken along cross-sectional lines III-III of FIG. 1;

FIG. 4 is a cross-sectional view of a coiled wire conductor forming a multi-filar conductor coil according to an embodiment of the present invention; and

FIG. 5 is a cross-sectional view of a coiled wire conductor forming a multi-filar conductor coil according to an embodiment of the present invention.

### DETAILED DESCRIPTION OF THE INVENTION

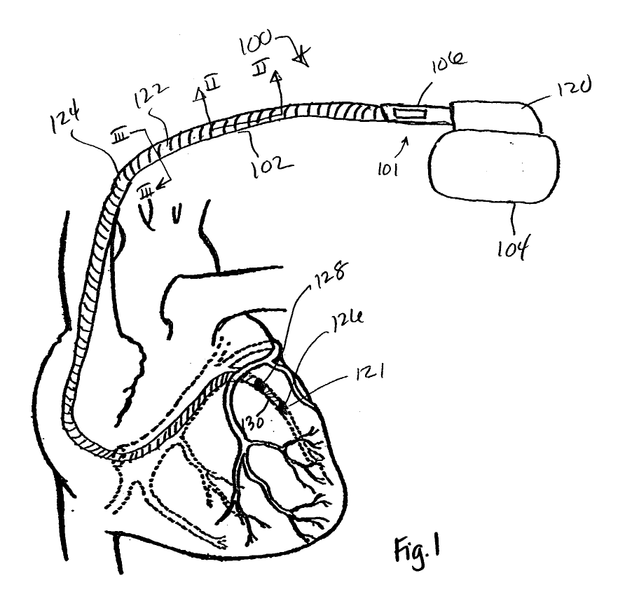

FIG. 1 is a schematic diagram of an exemplary implantable medical device in accordance with the present invention. As illustrated in FIG. 1, an implantable medical device 100 according to the present invention includes an implantable medical device lead 102 and an implantable medical device housing 104, such as an implantable cardioverter/defibrillator or pacemaker/cardioverter/defibrillator (PCD), for example, for processing cardiac data sensed through lead 102 and generating electrical signals in response to the sensed cardiac data for the provision of cardiac pacing, cardioversion and defibrillation therapies. A connector assembly 106 located at a proximal end 101 of lead 102 is insertable within a connector block 120 of housing 104 to electrically couple lead 102 with electronic circuitry (not shown) of housing 104.

Lead 102 includes an elongated lead body 122 that extends between proximal end 101 and a distal end 121 of lead 102. An outer insulative sheath 124 surrounds lead body 122 and is preferably fabricated of polyurethane, silicone rubber, or an ethylene tetrafluoroethylene (ETFE) or a polytetrafluoroethylene (PTFE) type coating layer. Coiled wire conductors in accordance with the present invention are positioned within lead body 122, as will be described in detail below. Distal end 121 of lead 102 includes a proximal ring electrode 126 and a distal tip electrode 128, separated by an insulative sleeve 130. Proximal ring electrode 126 and distal tip electrode 128 are electrically coupled to connector assembly 106 by one or more coil conductors, or filars extending between distal end 121 and proximal end 101 of lead 102 in a manner shown, for example, in U.S. Pat. Nos. 4,922,607 and 5,007,435, incorporated herein by reference in their entireties.

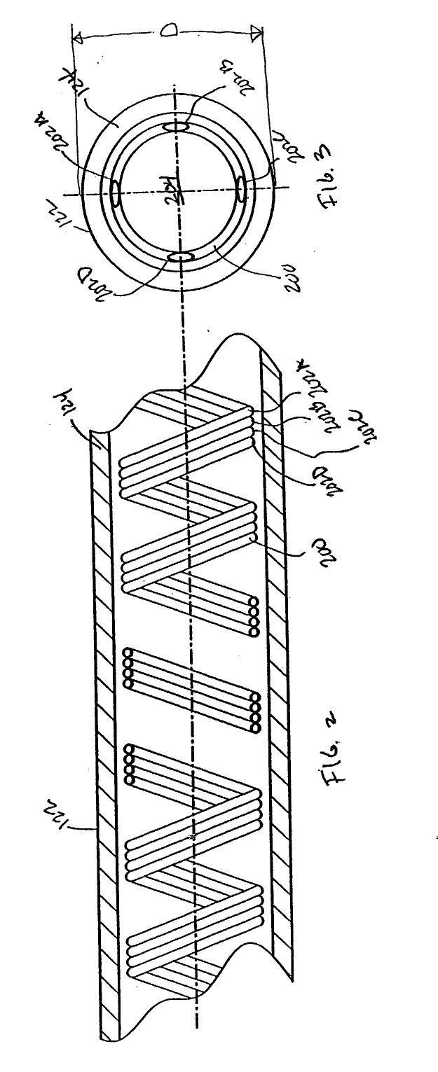

FIG. 2 is a cross-sectional view of a lead of an implantable medical device according to the present invention, taken along cross-sectional lines II-II of FIG. 1. As illustrated in FIG. 2, lead 102 of implantable medical device 100 includes a quadrifilar conductor coil 200 including four individual filars, or coiled wire conductors 202A, 202B, 202C and 202D extending within insulative sheath 124 of lead body 122. Coiled wire conductors 202A-202D electrically couple proximal ring electrode 126 and distal tip electrode 128 with connector assembly 106. It is understood that although the present invention is described throughout in the context of a quadrafilar conductor coil, having each of two electrodes electrically coupled to a connector assembly via two of the four individual coiled wire conductors, the present invention is not intended to be limit to application in a quadrafilar conductor coil. Rather, the lead conductor insulator of the present invention can be utilized in any conductor configuration, including the use of any number of conductor coils depending upon the number of desired electrodes, and would include the use of a single filar electrically coupling the electrode to the connector.

FIG. 3 is a cross-sectional view of a lead of an implantable medical device according to the present invention, taken along cross-sectional lines III-III of FIG. 1. As illustrated in FIGS. 2 and 3, each of the individual filars or coiled wire conductors 202A, 202B, 202C and 202D are parallel-wound in an interlaced manner to have a common outer and inner coil diameter. As a result, conductor coil 200 forms an internal lumen 204, which allows for passage of a stylet or guide wire (not shown) within lead 102 to direct insertion of lead 102 within the patient.

Alternately, lumen 204 may house an insulative fiber, such as ultrahigh molecular weight polyethylene (UHMWPE), liquid crystal polymer (LCP) and so forth, or an insulated cable in order to allow incorporation of an additional conductive circuit and/or structural member to aid in chronic removal of lead 102 using traction forces. Such an alternate embodiment would require insertion and delivery of lead 102 to a final implant location using alternate means, such as a catheter, for example. Lumen 204 may also include an insulative liner (not shown), such as a fluoropolymer, polyimide, PEEK, for example, to prevent damage caused from insertion of a style/guidewire (not shown) through lumen 204.

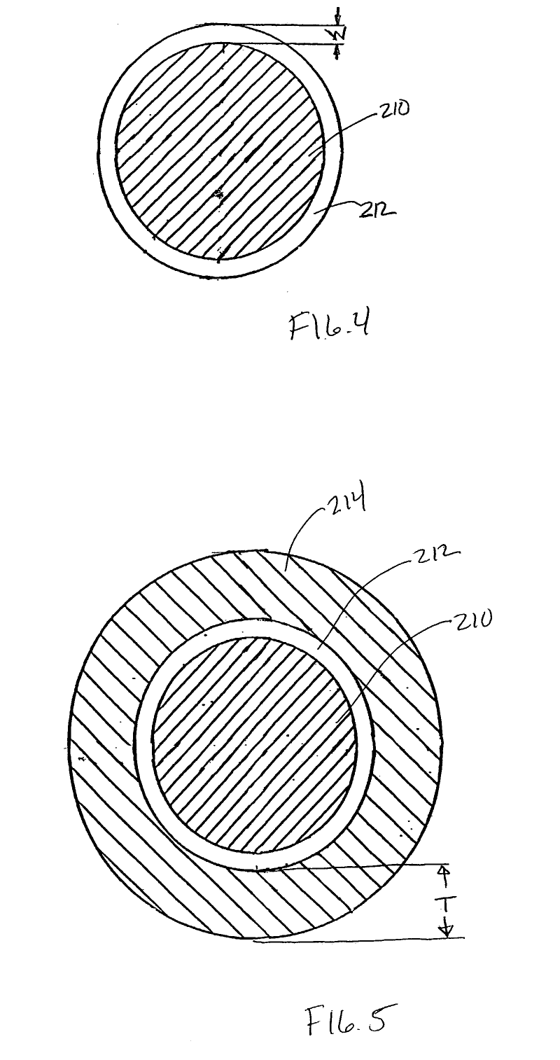

FIG. 4 is a cross-sectional view of a coiled wire conductor forming a multi-filar conductor coil according to a preferred embodiment of the present invention. As illustrated in FIG. 4, one or more of the individual coiled wire conductors 202A, 202B, 202C and 202D includes a conductor wire 210 surrounded by an insulative layer 212. According to the present invention, insulative layer 212 is formed of a hydrolytically stable polyimide, such as a Soluble Imide (SI) polyimide material, for example, (formerly known as Genymer, Genymer SI, and LARC SI) as described in U.S. Pat. No. 5,639,850, issued to Bryant, and incorporated herein by reference in it's entirety, to insulate conductor coils in implantable medical device leads. Such SI polyimide material is currently commercially available from Dominion Energy, Inc. (formerly Virginia Power Nuclear Services), for example. The thickness of the insulative layer 212 ranges from approximately 0.0001 inches up to approximately 0.0050 inches, forming a corresponding wall thickness W of the insulative layer 212. By utilizing the hydrolytically stable polyimide material as an insulative layer 212, the present invention provides an improved electrically insulating material that is hydrolytically stable in implantable (in vivo) applications.

According to the present invention, the insulative layer 212 is applied onto the conductor wire 210 in multiple coats to obtain a desired wall thickness W. The coating is applied in such a way to provide a ductile, robust insulative layer that enables a single filar, i.e., coiled wire conductor, or multiple filar, i.e., coiled wire conductors, to be wound into a single wound conductor coil 200 of sizes ranging from an outer diameter D (FIG. 3) of 0.010 inches to 0.110 inches. For example, according to the present invention, the coating process includes a solvent dip followed by an oven cure cycle to drive off the solvents. The multiple coating passes during the application of the insulative layer 212 onto the conductor wire 210 provides the ductility between layers that is needed to make the coated conductor wire 210 into a very tight wound conductor coil 200 and that can withstand the long term flex requirements of an implantable stimulating lead. As a result, the material is hydrolytically stable over time, and the process of applying the SI polyimide in thin coatings, through multiple passes, provides a ductile polyimide that can be wound into a conductor coil.

The use of the hydrolytically stable polyimide insulative layer 212 according to the present invention offers an exceptional dielectric strength and provides electrical insulation. Through flex studies on conductor coils coated with the SI polyimide, for example, the inventors have found that the insulative layer 212 also has high flex properties in regards to stimulating lead conductor coil flex testing. The SI coating in various wall thicknesses will remain intact on the coil filar until the coil filar fractures as seen in conventional conductor coil flex studies (reference 10 million to 400 million flex cycles at various 90 degree radius bends).

Conductor coils 200 (FIG. 2) according to the present invention, can include a single filar or multiple filars, with each filar being an individual circuit that could be associated with either a tip electrode, a ring electrode, a sensor, and so forth. In known lead designs, each lead utilizes one coil per circuit with a layer of insulation. The present invention enables the use of multiple circuits in a single conductor coil, resulting in a downsizing of the implantable medical device. For example, there is approximately a 40 to 50 percent reduction in lead size between known bipolar designs, which traditionally utilized an inner coil and inner insulation, outer coil and outer insulation, to a lead design having multiple circuits in a single conductor coil having the insulative layer 212 according to the present invention.

FIG. 5 is a cross-sectional view of a coiled wire conductor forming a multi-filar conductor coil according to a preferred embodiment of the present invention. The insulative layer 212 of the present invention can be utilized as a stand-alone insulation on a filer or as an initial layer of insulation followed by an additional outer layer as redundant insulation to enhance reliability. For example, according to an embodiment of the present invention illustrated in FIG. 5, in addition to conductor wire 210 and insulative layer 212, one or more of the individual coiled wire conductors 202A, 202B, 202C and 202D includes an additional outer insulative layer 214, formed of known insulative materials, such as ETFE, for example, to enhance reliability of the lead. According to the present invention, insulative layer 214 generally has a thickness T between approximately 0.0005 and 0.0025 inches, for example, although other thickness ranges are contemplated by the present invention. Since the outermost insulative layer, i.e., insulative layer 214, experiences more displacement during flex of lead 102 than insulative layer 212, it is desirable for insulative layer 214 to be formed of a lower flex modulus material than insulative layer 212, such as ETFE.

By utilizing the insulative layer 212 of the present invention, the stimulating lead is reduced in diameter, and is more robust in regards to mechanical flex and electrical insulation. The insulative layer 212 provides an extremely long-term flex-life performance associated with the ductility of the hydrolytically stable polyimide coating over conductor wires such as MP35N, used on conductor coils. These improved properties are related to the unique process of the multiple pass application of the hydrolytically stable polyimide. The resulting insulative layer 212 provides a highly reliable insulating and mechanically robust coating over implantable stimulating leads.

While an insulative layer formed only of ETFE tends to be susceptible to creep, insulative layer 212 of the present invention, which is formed of hydrolytically stable polyimide, is mechanically more robust, hydrolytically stable and possesses exceptionally dielectric properties, making the hydrolytically stable polyimide desirable for long-term implant applications. The use of a thin layer of hydrolytically stable polyimide coating on conventional MP35N alloy coil filars will also act as a protective barrier to reduce the incidence of metal induced oxidation seen on some polyurethane medical device insulations.

While a particular embodiment of the present invention has been shown and described, modifications may be made. It is therefore intended in the appended claims to cover all such changes and modifications, which fall within the true spirit and scope of the invention.

1. An implantable medical device, comprising:
a lead body extending from a proximal end to a distal end;
a plurality of conductors extending between the proximal end and the distal end of the lead body; and
an insulative layer positioned about the plurality of conductors, wherein the insulative layer is formed of a hydrolytically stable polyimide material.

2. The implantable medical device of claim 1, wherein the hydrolytically stable polyimide material is an SI polyimide material.

3. The implantable medical device of claim 1, wherein the insulative layer has a thickness of between approximately 0.0001 inches and approximately 0.0050 inches.

4. The implantable medical device of claim 1, wherein the insulative layer is positioned about the plurality of conductors in multiple coats to form multiple layers.

5. The implantable medical device of claim 1, wherein the plurality of conductors form a conductor coil having an outer diameter between approximately 0.010 inches and approximately 0.110 inches.

6. The implantable medical device of claim 1, wherein one or more of the plurality of conductors form a single circuit.

7. The implantable medical device of claim 1, further comprising a redundant insulative layer positioned about the plurality of conductors.

8. The implantable medical device of claim 7, wherein the redundant insulative layer is formed of a material having a flex modulus less than the insulative layer surrounding the plurality of conductors.

9. An implantable medical device, comprising:
a housing generating electrical signals for delivering therapy, the housing having a connector block;
a lead having a lead body extending from a proximal end to a distal end, the proximal end of the lead body being insertable within the connector block and electrically coupling the housing and the lead;
a plurality of conductors extending between the proximal end and the distal end of the lead body; and
an insulative layer positioned about the plurality of conductors, wherein the insulative layer is formed of a hydrolytically stable polyimide material.

10. The implantable medical device of claim 9, wherein the hydrolytically stable polyimide material is an SI polyimide material.

11. The implantable medical device of claim 9, wherein the insulative layer has a thickness of between approximately 0.0001 inches and approximately 0.0050 inches.

12. The implantable medical device of claim 9, wherein the insulative layer is positioned about the plurality of conductors in multiple coats to form multiple layers.

13. The implantable medical device of claim 9, wherein the plurality of conductors form a conductor coil having an outer diameter between approximately 0.010 inches and approximately 0.110 inches.

14. The implantable medical device of claim 9, wherein one or more of the plurality of conductors forms a single circuit.

15. The implantable medical device of claim 9, further comprising a redundant insulative layer positioned about the plurality of conductors.

16. An implantable medical device, comprising:
a lead body extending from a proximal end to a distal end;
a plurality of conductors extending between the proximal end and the distal end of the lead body; and
an insulative layer positioned about the plurality of conductors, wherein the insulative layer is formed of a hydrolytically stable polyimide material, and wherein the insulative layer is positioned about the plurality of conductors in multiple coats to form multiple layers and has a thickness of between approximately 0.0001 inches and approximately 0.0050 inches.

17. The implantable medical device of claim 16, wherein the hydrolytically stable polyimide material is an SI polyimide material.

18. The implantable medical device of claim 16, wherein the plurality of conductors form a conductor coil having an outer diameter between approximately 0.010 inches and approximately 0.110 inches.

19. The implantable medical device of claim 16, wherein one or more of the plurality of conductors form a single circuit.

20. The implantable medical device of claim 16, further comprising a redundant insulative layer positioned about the plurality of conductors.

21. The implantable medical device of claim 20, wherein the redundant insulative layer is formed of a material having a flex modulus less than the insulative layer surrounding the plurality of conductors.

22. An implantable medical device, comprising:
a housing generating electrical signals for delivering therapy, the housing having a connector block;
a lead having a lead body extending from a proximal end to a distal end, the proximal end of the lead body being insertable within the connector block and electrically coupling the housing and the lead;
a plurality of conductors extending between the proximal end and the distal end of the lead body; and
an insulative layer positioned about the plurality of conductors, wherein the insulative layer is formed of a hydrolytically stable polyimide material, and wherein the insulative layer is positioned about the plurality of conductors in multiple coats to form multiple layers and has a thickness of between approximately 0.0001 inches and approximately 0.0050 inches.

23. The implantable medical device of claim 22, wherein the hydrolytically stable polyimide material is an SI polyimide material.

24. The implantable medical device of claim 22, wherein the plurality of conductors form a conductor coil having an outer diameter between approximately 0.010 inches and approximately 0.110 inches.

25. The implantable medical device of claim 22, wherein one or more of the plurality of conductors form a single circuit.

26. The implantable medical device of claim 22, further comprising a redundant insulative layer positioned about the plurality of conductors.

27. The implantable medical device of claim 26, wherein the redundant insulative layer is formed of a material having a flex modulus less than the insulative layer surrounding the plurality of conductors.

28. An implantable medical device, comprising:
a lead body extending from a proximal end to a distal end;
a plurality of conductors extending between the proximal end and the distal end of the lead body; and
an insulative layer positioned about the plurality of conductors, wherein the insulative layer is formed of an SI polyimide material.

29. The implantable medical device of claim 28, wherein the insulative layer has a thickness of between approximately 0.0001 inches and approximately 0.0050 inches.

30. The implantable medical device of claim 29, wherein the insulative layer is positioned about the plurality of conductors in multiple coats to form multiple layers.

31. The implantable medical device of claim 30, wherein the plurality of conductors form a conductor coil having an outer diameter between approximately 0.010 inches and approximately 0.110 inches.

32. The implantable medical device of claim 31, further comprising a redundant insulative layer positioned about the plurality of conductors.

33. The implantable medical device of claim 32, wherein the redundant insulative layer is formed of a material having a flex modulus less than the insulative layer surrounding the plurality of conductors.

34. The implantable medical device of claim 33, wherein one or more of the plurality of conductors form a single circuit.
